(19) United States
(12) Patent Application Publication
(10) Pub. No.: US 2009/0017120 A1
(43) Pub. Date: Jan. 15, 2009

(54) Phase stable lecithin organogel composition

(75) Inventors: John Olin Trimble, Texarkana, TX (US); Christopher Michael Brisco, Wake Village, TX (US)

Correspondence Address: JACKSON WALKER LLP, 901 MAIN STREET, SUITE 6000, DALLAS, TX, 75202-3797, US

(73) Assignee: Humco Holding Group, Inc., Texarkana, TX (US)

(21) Appl. No.: 12/075,653

(22) Filed: Mar. 13, 2008

Related U.S. Application Data
(60) Provisional application No. 60/919,624, filed on Mar. 23, 2007.

(51) Int. Cl. A61K 9/10; A61P 43/00

(52) U.S. Cl. 424/485; 424/486

(57) ABSTRACT

A lecithin organogel composition used to deliver pharmaceutical products transdermally as well as a method for producing the lecithin organogel composition, which may contain up to 40% additive ingredients. Preferred embodiments of the invention may include lecithin organogel compositions which provide high penetrating power, which are ready-to-use, which have improved stability, which have a high uptake capacity for active drugs, and which do not grow mold if the gel becomes contaminated.

This application claims priority to U.S. Provisional Patent Application Ser. No. 60/919,624, entitled “PHASE STABLE LECITHIN ORGANOGEL COMPOSITION,” filed on Mar. 23, 2007, the entire content of which is hereby incorporated by reference.

### BACKGROUND

The present invention relates to a transdermal pharmaceutical delivery composition, including matrices of a lecithin gel, such as a lecithin organogel. In particular, this invention relates to compositions which comprise an internal oil phase containing optional non-ionic emulsifying agents, and an aqueous phase comprising gelling agents. Microscopically, these compositions maintain emulsion droplet integrity while macroscopically there is little or no phase separation.

### PLO Gels

Pluronic-lecithin-organogel (PLO) is a transdermal carrier used by pharmacists to deliver drugs through the skin when other routes of administration are not viable. PLO gel is non-irritating to the skin and is absorbed quickly. It is best used with drugs with molecular weights, preferably less than about 400. PLO gel may include isopropyl palmitate, soy lecithin, water, and pluronic F127.

Isopropyl palmitate is a non-oleaginous emollient with a high capacity for spreading.

Lecithin is a naturally occurring mixture of diglycerides of fatty acids linked to the choline ester of phosphoric acid. It is used as a penetration enhancer in compounding the PLO gel. It is a liquid at room temperature and may become solid upon cooling. It is normally stored at room temperature. Lecithins vary greatly in their physical form from semiliquids to powders. They are almost odorless and vary from brown to light yellow. They decompose at extreme pH's and are hygroscopic. They will oxidize and darken at high temperatures. Lecithin is usually stored at room temperature and protected from light. Refrigeration may cause the material to separate.

Pluronic F127 is a long chain polymer that has the unique property of being a solid at room temperature. It is a liquid when at refrigerated temperatures and becomes more viscous upon warming. It is normally stored at around 4° C. Poly(ethylene oxide)-poly(propylene oxide)-poly(ethylene oxide) triblock copolymers (PEO-PPO-PEO block copolymers) are widely used in diverse industrial applications (Alexandridis, 1997). PEO-PPO-PEO block copolymers have recently attracted considerable interest in the biotechnological and pharmaceutical industries for their unique surfactant abilities and low toxicity (Nace, 1996). Aqueous solutions of PEO-PPO-PEO block copolymers exhibit interesting temperature-induced aggregation phenomena as a result of the hydrophobic nature of the PPO block (Hvidt, 1994). PEO-PPO-PEO block copolymers exist in solution as dissolved monomers at low temperature and concentration but self-assemble at higher concentrations and temperatures into block copolymer micelles that form under conditions defined by the critical micelle concentration and the critical micelle temperature.

To form PLO gel, active ingredients are combined and triturated to like particle size. Powder is wetted with the smallest amount possible of levigating agent or solvent and mixed to form a smooth paste. Lecithin is added and mixed until smooth. A sufficient amount of pluronic is incorporated.

Previously, the addition of greater than 20% to 25% of additional ingredients to PLO gel would result in cracking of the gel. The current invention allows the addition of up to 40% by weight total of additional materials. Examples of additional materials are ibuprofen and ketoprofen.

In order to understand how a PLO gel can be used to deliver materials transdermally, it is important to first understand the barriers in the skin which prevent absorption into the skin. The skin is composed of three major components: the epidermis, the dermis, and the underlying subdermal tissue. The epidermis is composed of five different layers: stratum corneum; stratum lucidum; stratum granulosum; stratum spinosum; stratum basale. The stratum corneum is the most impermeable of these five layers. The stratum corneum can be compared to a brick wall. The stratum corneum consists of flattened cells imbedded in a lipid intercellular matrix just as a brick wall consists of bricks and mortar.

Without wishing to be bound by theory, two mechanisms for gel permeation into the skin have been proposed. One possible mechanism for gel permeation into the skin occurs by diffusion through the lipid intercellular matrix described above. Another proposed mechanism is that the PLO gel provides a slight disorganization of the skin allowing permeation of the gel and the active drug through the statum corneum. One thing that seems clear is that the lecithin component of the PLO has the ability to act as an amphoteric surfactant and enables many drugs to penetrate the dermal layer.

Topical agents have had relatively poor bioavailability in the past. With the advent of PLO gel the problem of bioavailability has been somewhat resolved. The PLO provides an adequate vehicle that permeates the stratum corneum. Lecithin is able to pass through the stratum corneum because it is a lipophilic substance. Both a drug and a hydrophobic medium can pass through the epidermis when the water-soluble drug is added to the hydrophobic substance. Bioavailability may range from about 10% to 60%.

Several different compositions for lecithin organogels have been described.

U.S. Pat. No. 5,176,916 to Yamanaka, et al., describes a plaster layer containing a medicinal ingredient, a hydrophobic polymer having a glass transition temperature of −65 degrees Celsius to 35 degrees Celsius, a percutaneous absorption-promoting agent, water, a hydrophobic polymer which is soluble or capable of swelling in water, and a medical adhesive which comprises a porous base layer in the plaster layer or in contact with the plaster layer, which enables stable release of the medicinal ingredient. The composition includes a larger amount of the hydrophobic polymer having a glass transition temperature of −65 degrees Celsius to 35 degrees Celsius than the hydrophobic polymer which is soluble or capable of swelling in water. The hydrophobic polymer having a glass transition temperature of −65 degrees Celsius to 35 degrees Celsius is present in an amount not less than 25% weight relative to the total amount of the ingredients.

U.S. Pat. No. 5,613,958 to Kochinke, et al., issued Mar. 25, 1997, describes a transdermal delivery system for the modulated administration of drugs. The drug delivery device comprises a backing; a drug reservoir containing the drug, a plasticizer-type enhancer, a solvent-type enhancer, and optionally, a gelling agent; a non-rate-controlling membrane; and an adhesive layer containing a plasticizer-type enhancer. This drug delivery system is particularly useful for the administration of tolerance-inducing drugs, for example, vasodilators, such as isosorbide dinitrate.

U.S. Pat. No. 5,665,378 to Davis, et al., issued Sep. 9, 1997, comprises capsaicin, a nonsteroidal anti-inflammatory agent, and pamabrom, a diuretic. The formulation is used to alleviate pain or discomfort in a mammal by being applied to the skin of the mammal thereby causing the active ingredients in the formulation to pass into and/or through the skin of the mammal. In a preferred embodiment of the present invention, the formulation is used in patch form for the treatment of the pain and discomfort associated with menstrual cramps, water retention (e.g., “bloating”) and/or muscular pain (e.g., muscular back pain).

U.S. Pat. No. 5,837,289 to Grasela, et al., issued Nov. 17, 1998, describes a composition and procedures for its formation and administration, which provide for a convenient, efficacious and simple transdermal administration of medications from a topically applied cream. No transmission through a membrane is involved. The composition incorporates at least two separate penetration enhancers which function synergistically to provide for rapid but controllable transport of the medication from the cream into the skin. The use of a plurality of penetration enhancers, at least one of which facilitates the separation of medication from the cream and at least a second of which alters the structure of the outer layers of skin, particularly the stratum corneum, enhances migration of the drug through the stratum corneum.

U.S. Pat. No. 5,885,597 to Botknecht, et al., issued Mar. 23, 1999, discloses a topical composition for relieving pain in a person in need of such relief, consisting essentially of an effective amount of a combination of at least one corticoid analgesic, at least one arylpropionic acid type analgesic, and at least one p-aminobenzoic acid ester type local anesthetic; an amount effective in enhancing the effectiveness in relieving pain of the combination of capsaicin, and an amount effective to increase the transmission thereof of through the skin of at least one phospholipid and at least one polyoxyethylenepolyoxypropylene copolymer.

U.S. Pat. No. 6,290,986 to Murdock, et al., issued Sep. 18, 2001, provides a method and composition for transdermal delivery of pharmaceuticals or combinations of pharmaceuticals. The pharmaceuticals are delivered using a matrix of a lecithin gel such as a lecithin organogel. A number of psychopharmaceuticals can be used including fluoxetine, sertraline, carbamazepine, paroxetine, amitriptyline, trazadone, venlafaxine, propranolol, buproprion, valproic acid, nefazodone, ketoprofen, gabapentin, piroxican, doxepin, guaifenesin, pemoline and doxepin and combinations.

Gallipot, Inc. manufactures two components of PLO which previously required a somewhat lengthy preparation process. These products are trademarked as Lipoil and Polox. Polox is available in 20% and 30% and is a reconstituted aqueous gel made from Pluronic F127NF and containing preservatives. It is a liquid at refrigerated temperatures and becomes more viscous upon warming. It is usually stored at about 4 degrees Celsius. Lipoil is a 50:50 eutectic mixture of solid lecithin and liquid isopropyl palmitate. It is used as a penetration enhancer in compounding the PLO gel. It is a liquid at room temperature and may become solid upon cooling. It is stored at room temperature.

J.A.R. Pharmaceuticals, Ltd. manufactures Phlogel. Phlogel Hydrophilic is a PLO gel. Phlogel Organic is a lecithin organogel. Phlogel Ultra is a proprietary premixed ready-to-use lecithin organogel which eliminates pluronic from the gel.

Maxima Pharmaceuticals, Inc. manufactures Diffusimax. The Diffusimax kit is supplied as separate hydrophilic Diffusimax B and lipophilic Diffusimax A, which are components for making PLO gel. Diffusimax B is refrigerated prior to use so that it is in liquid form for mixing. Diffusimax A is warmed to room temperature prior to use. Both components are stable for 12 months at room temperature if unopened. Mold may grow if the gel becomes contaminated. Diffusimax Pre-Mixed Cream is supplied as a pre-mixed cream. It is stable for 12 months at room temperature. Stability is reduced once the container is opened and medications are added. Mold may grow if the gel becomes contaminated.

Medisca, Inc. manufactures Lipmax (lecithin and isopropyl palmitate), Mediflo Pre-Mixed PLO Gel, a PLO Gel Kit, and Pluronic Gel 20% (F127).

Transderma Pharmaceuticals, Inc. manufactures PLO Premixed Gel, PLO Ultramax Gel, and PLO Kit. PLO Premixed Gel has no pluronic surfactant. PLO Ultramax Gel answers the need for ready-to-use vehicles to incorporate higher percentages of active drugs without disturbing the elegant nature or the efficacy of the gel. PLO Kit is packed with organic phase, hydrophilic phase, and ethoxy diglycol solvent.

Almost all of the above matrices of lecithin gels achieve some tissue-levels of active compounds, reducing blood-level related side effects. Most of these matrices of lecithin gels require compounding time. Many of the commercial matrices of lecithin gels do not remain uniform and usable for long periods of time or under varying storage conditions. They do not satisfy the demand of varying percentages of active compounds as well as pharmacists' demands for time-efficient and uniform compounding. The present invention overcomes many of these problems.

### Ethanol/Isopropyl Palmitate Systems

PLO gels employing isopropyl palmitate and ethanol are noted to be effective for topical application of non-steroidal anti-inflammatory drugs, agents to target neuropathy, and systemic hormone drugs. Both ethanol and isopropyl palmitate act as permeation enhancers on the skin. Active drug steady-state permeation rates increase as the volume fraction of ethanol in donor solutions is increased.

Ethanol has been used very successfully as a permeation enhancer in a variety of commercial transdermal systems. Ethanol is the most commonly used alcohol as a transdermal penetration enhancer. One possible drawback could be skin irritation induced by high dose ethanol. Without wishing to be bound by theory, pure ethanol-enhanced permeation of solutes across the skin in vitro is thought to be a result of the extraction of skin lipids by ethanol. Many researchers have investigated the mechanism of the enhancing effect of ethanol on skin permeability. Some have demonstrated an ethanol concentration dependent enhancement mechanism. Low concentrations of ethanol affect only the lipid pathway and high concentrations of ethanol affect both the lipid and polar pathways.

The mechanism of action of isopropyl palmitate is poorly understood despite its well established use in the pharmaceutical industry. There is some evidence that isopropyl palmitate intercalates into the structured lipids of the horny layer where they disrupt the packing.

The increase in drug content associated with the increased ethanol fraction means that more of the drug is available to be partitioned with the vehicle system into the skin. This can lead to a higher active drug flux values seen with increased volume ratios of ethanol. The ethanol/isopropyl palmitate system may increase the active drug solubility in the stratum corneum by increasing the partitioning of the drug.

### Preparation of PLO Gel

PLO gel may be prepared by mixing an oil phase with an aqueous phase using a high-shear mixing method. Oil Phase: charge kettle with Lecithin 50% Solution; install mixer; turn on the mixer; heat to 65°-75° C.; with mixing, add Arlacel 165 and Cremophor GS 32; mix for 15 minutes or until dissolved; lower the sweep mixer and add Poloxamer 407 30% Solution with mixing. Water Phase: in tank equipped with mixer, add Purified Water; turn on the mixer; heat to 80° C.; add Potato Starch and mix until dissolved. Emulsion Phase: transfer the Water Phase to the Oil Phase in kettle with mixing; with mixing, add SD-40B Alcohol, Germaben IIE, and Simulgel NS; mix for 1 hour; mill at 0.003″ to a storage vat.

A PLO base is composed of pluronic gel and lecithin. A gel is a two-phase colloidal system containing a solid and a liquid phase. Gels formed with pluronic are liquid at cold temperatures and undergo a phase change when the temperature is elevated. 20% w/w of Pluronic F127 retains the gel structure from about 20° C. to about 70° C. This characteristic makes it useful in pharmaceutical compounding because it can be drawn into a syringe for accurate dose measurement when it is cold. The degree of viscosity of the pluronic gel is dependent on the ratio of pluronic to water.

The oil phase may be prepared by mixing lecithin and isopropyl palmitate and allowing the mixture to stand overnight to ensure complete dissolution. The role of organic solvent in providing the desired solvent action onto the lecithin molecules is much emphasized. A large variety of organic solvents are able to form gel in the presence of lecithin. Isopropyl palmitate is of particular interest for topical applications of lecithin organogels. This has been attributed to its skin penetration enhancing property as well as its biocompatible and biodegradable nature.

The aqueous phase may be prepared by adding Pluronic F127 to ice cold water and agitating periodically to ensure complete dissolution. Pluronic is a bifunctional block copolymer surfactant consisting of ethylene oxide and propylene oxide terminating in a primary hydroxyl group. Pluronic gels may be formed by hydrogen bonding by attraction of the surfactant ether oxygen atoms with water protons in aqueous pluronic systems.

Pluronic is a reverse thermal gel and its viscosity increases with higher temperatures. Alteration of the component concentrations in any way may change the bioavailability of active drugs. Some organic solvents may weaken gel strengths or water-insoluble organic materials may increase gel strengths. Inorganic salts and strong electrolytes usually soften gels. The incorporation of additional surfactants may weaken gel strengths by competing with the hydrogen-bonding lattice.

Dispersion of a hydrophilic drug in the aqueous phase is conducted by dissolving the drug in a small quantity of water. Hydrophilic drugs include the muscle relaxant, cyclobenzaprine; the neuropathy drug, clonidine; antiemetics, dimenhydrinate, metaclopramide and scopolamine; systemic analgesics, hydromorphone and morphine sulfate. Hydrophilic drugs have an uptake capacity of about 20% to about 25%.

Dispersion of a lipophilic drug in the oil phase is conducted by mixing the drug with alcohol or propylene glycol. Lipophilic drugs include the non-steroidal anti-inflammatories, diclofenac, ibuprofen, ketoprofen, indomethacin and naproxen; muscle relaxants, baclofen and buspirone; neuropathy drugs, capsaicin, amitriptyline, gabapentin, phenyloin and ketamine; the antiemetic, dexamethasone; the systemic analgesic, acetaminophen; systemic hormones, progesterone and testosterone. Lipophilic drugs have an uptake capacity of about 5% to about 10%.

### Charateristics of PLO Gel

Pre-mixed PLO as well as PLO that is compounded with a medication must be stored at room temperature. PLO prescriptions will become thin or runny if patients inadvertently expose them to cool temperatures.

Pre-mixed PLO has a shelf life of approximately twelve months. PLO prescriptions are stable for about six months once compounded with drugs.

The stability of topical gel formulations containing active drugs and 20% w/w pluronic as a gel-forming agent has been determined at three different temperatures, 6° C., 20° C. +/−2° C. and 45° C. A w/w percentage refers to the number of grams of solute that are contained in 100 g. of solution. All formulations are stable at 20° C. +/−2° C. Storing gels at 6° C. results in the precipitation of active drugs. The viscosity decreases as storage time increases. The effect of storage temperatures on the viscosity is much less than the effect of storage time (Shawesh A., 2002).

The effects of excipients on the viscosity of topical gel formulations containing active drugs and 20% w/w pluronic have been also been studied. Increasing the amount of hexylene glycol or polyethylene glycol gave more viscous gels. The difference in viscosities was explained by the changes in the gel compositions. The results indicate that the excipients influence the physical characteristics of the gels.

A rheological study of 10% to 35% pluronic aqueous sols and of mixed gels containing 10% to 17.5% pluronic has been conducted at 15° C. to 35° C. The viscosity of pluronic sols increases with an increase in temperature and the mixed gels have thermoreversible properties. The viscosity of mixed gels is higher than that of the pluronic sols containing only pluronic because of the increase in total polymer concentration. The viscosity of mixed gels containing un-neutralized Noveon is lower than that of the neutralized mixed gels (Timaksiz, F. 2005).

Effects of gel additives on thermodynamic properties of phase transitions at gelation and gel melting have been reported on aqueous pluronic gels prepared by cold method containing about 20% to 24% w/w pluronic. By definition, a w/w percentage refers to the number of grams of solute that are contained in 100 g. of solution. Gelation decreases and gel melting increases with pluronic concentration. Gelation range narrows with sorbitol and polyethylene glycol (PEG). Suppression of gel melting is significantly higher than gelation with both the additives. Thermodynamic properties of pluronic gels are significantly altered with polymer concentration and water-soluble formulation additives.

Aqueous pluronic gels with active drugs containing different concentrations of additives like inorganic salts and polyethylene glycol have been obtained. Increase of the pluronic concentration increases the viscosity of the gels and changes the releasing process of active drugs from the gels. The sol-gel transition temperature is decreased by increasing the pluronic concentration and by the presence of additives like inorganic salts and polyethylene glycol (PEG).

Topical pluronic gel preparations of ketoprofen have been reported. The gels are chemically stable for eight months at room temperature and under refrigeration.

The stability of indomethacin in pluronic gels has been evaluated. The time to 10% indomethacin degradation was 2.7 years in 20% wt/vol gels at pH 7 and 20° C. By definition, a w/v percentage refers to the number of grams of solute that are contained in 100 ml. of solution.

Phenyloin topicals for the treatment of skin wounds and pressure ulcers have been reported. A use period of six months is suggested.

### PLO Gel in the Delivery of Therapeutic Agents

A wide range of drugs actives have been incorporated within PLO for transdermal delivery, including hormones, non-steroidal anti-inflammatory drugs, selective seretonin reuptake inhibitors, antipsychotic drugs, and calcium channel blockers.

The skin is a good barrier to active drug permeation and active drug flux is known to be low. Active drug absorption following application to the skin is so low that only a few active drugs have been formulated for transdermal delivery. An ideal active drug for transdermal delivery is: (a) a potent chemical with a daily dose of a few milligrams; (b) a small molecule; (c) a drug that has a high lipid solubility and reasonable water solubility; and (d) non-irritating and non-sensitizing to the skin.

Transdermal delivery has quickly gained acceptance as a unique delivery route providing an alternative to existing oral therapeutic regimens. The skin is a virtually impermeable barrier to most environmental and synthetic compounds. Some highly potent and lipophilic compounds may permeate the skin to deliver therapeutically relevant amounts of active drugs. Some therapeutic advantages to transdermal delivery include: avoidance of local gastrointestinal toxicities; avoidance of first-pass metabolism; concentration of drug at localized sites where it is needed.

Transdermal delivery can be a superior alternative for active drugs which are potent and relatively hydrophobic. Transdermal delivery is a generally inefficient process to deliver active drugs for systemic purposes because only a small percentage of the administered dose enters the circulation. There is an advantage in transdermal delivery for active drugs which exert their action locally because of the high incidence of side-effects after oral administration which is directly correlated with blood concentrations. Low concentrations of potent active drugs in the bloodstream likewise minimize side effects.

The absolute bioavilability of a compound delivered transdermally is generally less than that delivered orally unless the compound is highly metabolized in the liver. Preliminary studies have indicated that the bioavailability of active drugs applied topically is approximately 5% to 10% of an equivalent oral dose.

Pluronic lecithin organogel is an organic mixture that supports mold growth. Anti-microbial preservatives are often added to pluronic lecithin organogel in order to supplement intrinsic anti-microbial activity. One such preservative is sorbic acid at 0.2 percent w/w, which is often added to pluronic lecithin organogel as a preservative. The development of pluronic lecithin with adequate anti-microbial activity may prevent the problems that could occur from microbial contamination or proliferation during storage. The United States Pharmacopeia (USP) details guidance on the performance and interpretation of preservative efficacy testing.

Germaben IIE (Diazolidinyl Urea, Methylparaben, Propylparaben) is a clear, viscous, liquid preservative system designed for emulsion systems with oil phases greater than 25%. It includes a total concentration of 20% parabens predissolved in Propylene Glycol for easy and convenient addition to cosmetic formulations. It minimizes the difficulties associated with incorporating solid parabens. It is a complete broad spectrum antimicrobial preservative system that is effective against Gram-positive and Gram-negative bacteria, yeast, and mold.

SD 40B Alcohol 190 Proof kills harmful bacteria, such as streptococcus, salmonella, staphylococcus, E. coli and shigella.

A number of thickening and stabilizing agents are available for use in transdermal delivery systems. Polaxamer 407 is used as a co-emulsifier and consistency enhancer in creams and liquid emulsions. It is used as a solubilizer for certain active drugs as well as for essential oils in pharmaceutical and cosmetic formulations. It is suitable for the formulation of active drugs that show reduced solubility as a result of neutralization, as well as being suitable as a stabilizer for topically and orally administered suspensions.

The addition of electrolytes to PLO reduces the gel formation temperature as well as the viscosity and pour point. Alcohols increase the gel formation temperature. Anionic surfactants may inhibit gel formation. Low pH values affect both the gel formation temperature and the viscosity.

An acid-stable self-emulsifying glycerol monostearate such as glyceryl stearate or polyethylene glycol (PEG) 100 stearate may be used as an emulsifier for creams and lotions that are rich in non-polar oils and waxes, and will facilitate the addition of acidic actives and salts. Emulsions made with glycerol stearate and PEG 100 stearate can be pH adjusted to between 4.5 to as high as 9.

Structure solanaceis a novel raw material that performs as a Theological additive. It provides thickening properties and emulsion stabilization while leaving excellent and long lasting after feel. It functions at low pH to provide stable viscosity and pH over time.

### SUMMARY

The present invention relates to a transdermal pharmaceutical delivery composition, including matrices of a lecithin gel such as a lecithin organogel. In particular, this invention relates to compositions which may comprise an internal oil phase containing optional non-ionic emulsifying agents, and an aqueous phase comprising gelling agents.

The current invention comprises a lecithin organogel composition which could be used to deliver pharmaceutical products transdermally. The invention further comprises a method for producing the lecithin organogel composition, which may contain up to 40% additive ingredients.

Preferred embodiments of the invention may include lecithin organogel compositions which provide high penetrating power, which are ready-to-use, which have improved stability, which have a high uptake capacity for active drugs, and which do not grow mold if the gel becomes contaminated.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a temperature sweep of a standard lecithin gel (“the Standard PLO Gel”) and a preferred embodiment of the invention (“the Humco PLO Gel”); and

FIG. 2 shows a stress sweep performed at 45° C. for a standard lecithin gel (“the Standard PLO Gel”) and a preferred embodiment of the invention (“the Humco PLO Gel”).

### DETAILED DESCRIPTION OF PREFERRED EMBODIMENTS

One aspect of the current invention pertains to a lecithin organogel composition which may be used to deliver pharmaceutical products transdermally. The invention further comprises a method for producing the lecithin organogel composition, which may contain up to 40% additive ingredients. Preferred embodiments of the invention may include lecithin organogel compositions which provide high penetrating power, which are ready-to-use, which have improved stability, which have a high uptake capacity for active drugs, and which do not grow mold if the gel becomes contaminated.

### Composition

A preferred embodiment of the lecithin organogel composition comprises a non-ionic emulsifying agent, preferably an oil-in-water emulsifier, most preferably a non-ionic emulsifying agent with a hydrophile-lipophile balance (HLB) value is from about 8 to about 18. HLB is calculated by the formula:

HLB=20(1−S/A)

wherein
S=sap. number of the ester
A=acid number of the recovered acid

Examples of non-ionic emulsifying agents may include polyglyceryl 1-3 distearate, a mono alkyl phosphate, a sorbitan ester, a polyethylene glycol oil/wax, a poly(ortho-ester)-fatty alcohol, a polyethylene glycol-stearate, a polysorbate, or a mixture thereof. The non-ionic emulsifying agent may be present in a concentration range of 0.5% to 3.5%, preferably 1.0% to 3.0%, most preferably 1.5% to 2.5%.

Oil-in-water emulsifiers comprise polyoxyethylene derivatives of sorbitan esters and have higher HLB numbers and they are also water soluble. Because of their water soluble character, they will cause the water phase to predominate and form an o/w emulsion.

This embodiment of the invention further comprises a thickening polymer, preferably an aqueous-alcoholic soluble thickener, most preferably aqueous-alcoholic soluble thickener with a solubility in from about 1 percent to about 30 percent of alcohol. Examples of thickening polymers include xanthan gum, acrylate/C₁₀-C₃₀ alkyl acrylate crosspolymer, hydroxypropylcellulose, acrylamide/sodium acryloyldimethyltaurate copolymer, isohexadecane and polysorbate 80, or a mixture thereof. The thickening polymer may be present in a concentration range of 1.0% to 8.0%, preferably 2.0% to 7.0%, most preferably 3.0% to 6.0%.

This embodiment further comprises a synthetic copolymer of ethylene oxide and propylene oxide, such as Poloxamer 407 (Chemical nature: Polyoxyethylene polyoxypropylene block copolymer, INCI name: Poloxamer 407, CAS No.: 9003-11-6) The synthetic copolymer of ethylene oxide and propylene oxide may be present in a concentration range of between about 1 and about 25 weight percent, preferably between about preferably 5 to 20 weight percent, most preferably 10 to 15 weight percent. Weight percent is calculated by dividing the weight of the ingredient by the total weight of the composition.

This embodiment may further comprise a lecithin content between about 1 and about 20 weight percent, preferably 5 to 15 weight percent (please list preferred range), most preferably 7 to 13 weight percent (please list most preferred range)

If desired, an acid-stable self-emulsifying glycerol monostearate such as glyceryl stearate or polyethylene glycol (PEG) 100 stearate may be used in the present invention. The glyceryl stearate may be present in a concentration range of 1.0 to 6.0 weight percent, preferably 2.0 to 5.0 weight percent, most preferably 3.0 to 4.0 weight percent.

If desired, a thickener such as, Cellulose, Xanthan Gums, Guar Gums, Vinyl Polymers or Simulgel NS (Chemical nature: Hydro swelling droplet polymer, INCI name: Hydroxyethyl acrylate/sodium acryloyldimethyltaurate copolymer and squalane and polysorbate 60, CAS No.: 111286-86-3/111-01-3/9005-67-8) may be used in the present invention. The thickner will most preferably be effective in an acidic medium as well as in an alkaline or oxidizing medium and thicken aqueous media over a wide pH range. The thickener may have synergy with pluronic to thicken in the presence of electrolytes. The thickener may be present in a concentration range of 1.0 to 6.0 weight percent, preferably 2.0 to 5.0 weight percent, most preferably 3.0 to 4.0 weight percent.

If desired, structure solanace may be used in the present invention. The structure solanace (Chemical nature: Carbohydrate, INCI name: Potato starch modified, CAS No.: Proprietary—National Starch and Chemical Company) may be present in a concentration range of 0.5 to 3.5 weight percent, preferably 1.0 to 3.0 weight percent, most preferably 1.5 to 2.5 weight percent.

If desired, an antimicrobial agent may be included in the composition, such as Imidazolidinyl Urea, Iodopropynyl Butylcarbamate, Sodium Hydroxymethylglycinate, Phenoxyethanol, Ethylparaben, Butylparaben or Germaben IIE (Chemical nature: Bacteria, yeast, and mold antimicrobial, INCI name: Propylene glycol and diazolidinyl urea and methylparaben and propylparaben, CAS No.: Proprietary—International Specialty Products).

The antimicrobial agent may be present in a concentration range of 0.5 to 1.0 weight percent, preferably 0.6 to 0.9 weight percent, most preferably 0.7 to 0.8 weight percent.

If desired, an antiseptic may be included in the invention, Benzalkonium Chloride, Benzethonium Chloride, Camphorated Metacresol, Camphorated Phenol, Eucalyptol, Hexylresorcinol, Hydrogen Peroxide, Iodine, Isopropyl Alcohol, Menthol, Methylbenzethonium Chloride, Methyl Salicylate, Phenol, Povidone-Iodine, Thymol or SD-40B (Chemical nature: 95% alcohol denatured with denatonium benzoate and tert-butyl alcohol, INCI name: SDA 40B, CAS No.: 64-17-5/3734-33-6/75-65-0/7732-18-5). The antiseptic agent may be present in a concentration range of 1 to 27 weight percent, preferably 9 to 18 weight percent, most preferably 12 to 15 weight percent.

### Methods

The lecithin organogel composition may be prepared be blending the proper amounts and ratios of all the required ingredients together. This lecithin organogel can later be used to dissolve active drugs to make the final prescription gel composition.

One embodiment of the invention would include preparation as follows:

Water Phase:
Into a glycol-jacketed tank, add Purified Water, USP. Turn on scraper at 5 RPM. Add Pluronic F127. Chill to 5-10° C. (41-50° F.). Increase scraper speed to 10 RPM and continue mixing for twenty-four (24) hours. Add SD 40B Alcohol 190 Proof. Add Potato Starch. Add Germaben IIE. Continue mixing for one (1) hour.

Oil Phase:
Into a 100-200 gallon heat-jacketed tank, add Isopropyl Palmitate. Add Lecithin. Without mixing, allow to dissolve for twenty-four (24) hours. Turn on mixer at 25 RPM. Add Arlacel 165. Add Cremophor GS32. Heat to 75-80° C. (167-176° F.).

Emulsion Phase:
Turn on WATER PHASE mixer at 25 RPM. Add OIL PHASE to WATER PHASE. Add Simulgel NS. Increase scraper speed to 10 RPM and mix for 15 minutes.

### Example 1

| Ingredient | Amount | |
|---|---|---|
| Purified Water (8.312 lb/gal @ 25 C.) | 38.4712 | w/w |
| Lecithin | 8.5900 | w/w |
| Isopropyl Palmitate | 8.5900 | w/w |
| Pluronic F127 | 13.7500 | w/w |
| Simulgel NS | 3.0000 | w/w |
| Arlacel 165 | 1.0800 | w/w |
| Cremophor GS32 | 0.7500 | w/w |
| Germaben IIE | 1.0000 | w/w |
| Potato Starch | 0.6000 | w/w |
| SD 40B Alcohol | 24.1688 | w/w |
| 190 Proof | | |

This embodiment of the invention is prepared as follows:

Water Phase
Into a glycol-jacketed tank, add Purified Water, USP. Turn on scraper at 5 RPM. Add Pluronic F127. Chill to 5-10° C. (41-50° F.). Increase scraper speed to 10 RPM and continue mixing for twenty-four (24) hours. Add SD 40B Alcohol 190 Proof. Add Potato Starch. Add Germaben IIE. Continue mixing for one (1) hour.

Oil Phase
Into a 100-200 gallon heat-jacketed tank, add Isopropyl Palmitate. Add Lecithin. Without mixing, allow to dissolve for twenty-four (24) hours. Turn on mixer at 25 RPM. Add Arlacel 165. Add Cremophor GS32. Heat to 75-80° C. (167-176° F.).

Emulsion Phase
Turn on WATER PHASE mixer at 25 RPM. Add OIL PHASE to WATER PHASE. Add Simulgel NS. Increase scraper speed to 10 RPM and mix for 15 minutes.

An ATS RheoSystems/Reologica Instruments Stresstech HR with 30 mm parallel plate geometry was used for dynamic properties characterization of a preferred embodiment of the lecithin organogel composition. The geometry was chosen based on physical properties of the samples.

A temperature sweep at a stress of 100 Pa from 20° C. to 100° C. at a rate of 2° C./minute was performed on both the current invention (“the Humco PLO Gel”) and a standard lecithin organogel (“the Standard PLO Gel”). Temperature accuracy was preserved at a set point of +/−0.1° C. The samples were loaded with a constant force of 10.0 N using the rheometer's differential pressure quantitative normal force sensor. This was done to ensure each sample had a comparable loading history. It was important to control the loading force with high degree of precision to protect the integrity of the material internal structure. A gap of 1.0 mm was used for all studies. All samples were trimmed at 50 μm above the target gap to avoid any edge effects. 180 s of equilibrium time was provided to ensure the equilibrium structure had been acquired before running the temperature sweep.

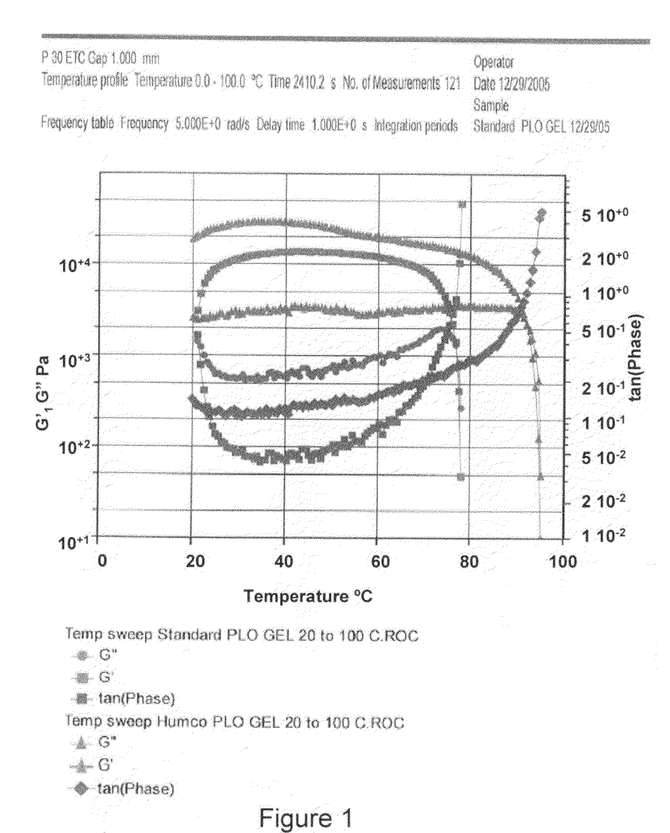

The data has been plotted for storage and loss modulus and tan phase versus temperature (FIG. 1).

The Humco PLO Gel sample shows higher elastic modulus and a tan delta value almost invariant with temperature up to 88° C. where the sample internal structure is broken down by heat. The Standard PLO Gel sample shows lower elastic modulus and a tan phase value with strong temperature dependence and a structure breakdown at 75 degrees Celsius (FIG. 1).

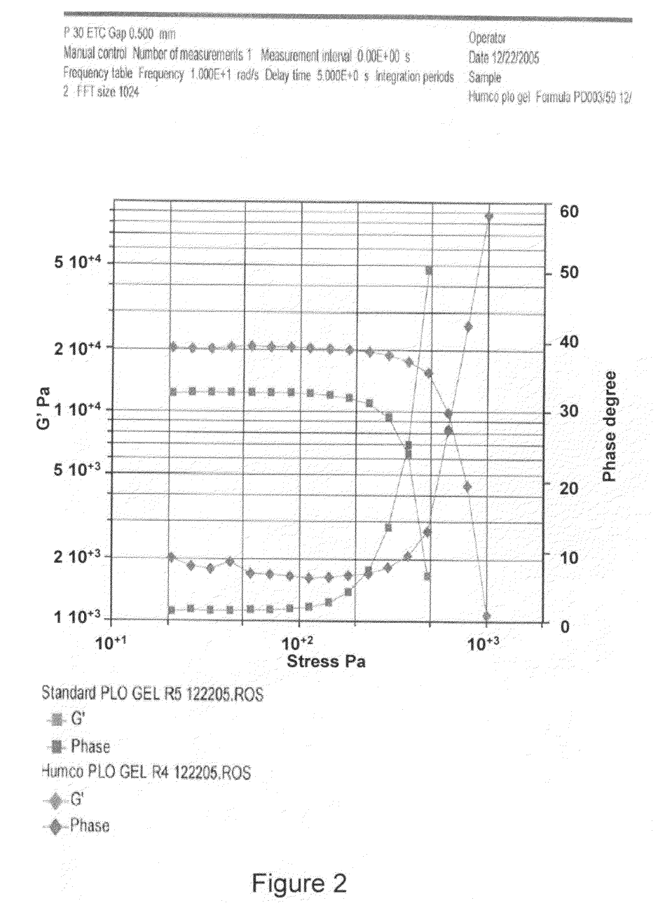

A stress sweep was performed at 45 degrees Celsius for both the Humco PLO Gel and the Standard PLO Gel and the resulting elastic modulus and phase are plotted versus stress (FIG. 2). The HUMCO sample maintains the integrity of its structure to higher applied stress values.

The stability of dispersions can be routinely measured by rheological techniques per Reboa et al. Stability is directly related to the temperature/frequency dependence on the tan phase values and the breadth of the LVE determined by a stress sweep curve. The Humco PLO Gel sample has superior stability due to its ability to maintain its structure to higher temperature, its invariant tan phase with temperature, and its broader LVE region. LVE is Linear Visco-Elastic. The LVE is analogous to the linear portion of a stress/strain curve or the region where the material exhibits complete recovery.

Table 1 shows a plate count experiment (total aerobic microbial count). The sample was pipetted onto each of two sterile petri dishes. Soybean-Casein Digest Agar Medium that was previously melted and cooled to approximately 45° C. was promptly added to each dish. The petri dishes were covered and the sample mixed with the agar by tilting or rotating the dishes. The petri dishes were inverted and incubated at 33° C. for 48 hours. The plates were examined for growth and the number of colonies counted.

TSB (test for Staphylococcus and Pseudomonas aeruginosa)-Staphylococcus aureus and Pseudomonas aeruginosa/Fluid Soybean-Casein Digest Medium was incubated at 33° C. for 24 hours. The sample was inoculated with 100,000 to 1,000,000 cfu per mL and incubated at 25° C. for 28 days. The medium was examined for growth and an inoculating loop was used to streak a portion of the medium on the surface of Vogel-Johnson Agar Medium and of Cetrimide Agar Medium. The sample met the requirements for Staphylococcus aureus and Pseudomonas aeruginosa of not less than 2.0 log reduction from initial count at 14 days and no increase from the 14 days' count at 28 days.

Lactose (test for Escherichia coli)—Escherichia coli/Fluid Lactose Medium was incubated at 33° C. for 24 hours. The sample was inoculated with 100,000 to 1,000,000 cfu per mL and incubated at 25° C. for 28 days. The medium was examined for growth and an inoculating loop was used to streak a portion of the medium on the surface of MacConkey Agar Medium. The sample met the requirements for Escherichia coli of not less than 2.0 log reduction from initial count at 14 days and no increase from the 14 days' count at 28 days.

Sab-dex (total combined molds and yeasts count)—Aspergillus niger/Sabourand Dextrose Agar Medium or Potato Dextrose Agar Medium was incubated at 25° C. for 5 days. The sample was inoculated with 100,000 to 1,000,000 cfu per mL and incubated at 25° C. for 28 days. The plates were examined for growth and the number of colonies counted. The sample met the requirements for Aspergillus niger of no increase from the initial calculated count at 14 and 28 days.

TABLE 1
Microbial Challenge Testing

| Microbial | | |
|---|---|---|
| Challenge | | |
| Testing | USP <51> | 28 Day |
| Lactose | Pass | Pass |
| Plate count | <100 cfu/g | Pass |
| Sab-dex | <100 cfu/g | Pass |
| TSB | Pass | Pass |

### Example 2

A preferred embodiment of the invention for increased amounts of lipophilic drug actives was prepared containing the following ingredients:

| Ingredients | Amount | |
|---|---|---|
| Purified Water | 6.3879% | w/w |
| Lecithin 50% in Isopropyl Palmitate | 17.1800% | w/w |
| Polaxamer 407 30% in Purified Water | 45.8333% | w/w |
| Simulgel NS | 3.0000% | w/w |
| Glyceryl Stearate & PEG 100 Stearate | 1.0800% | w/w |
| Polyglyceryl 1-3 Distearate | 0.7500% | w/w |
| Germaben IIE | 1.0000% | w/w |
| Structure Solanace | 0.6000% | w/w |
| SD 40B Alcohol 190 Proof | 24.1688% | w/w |

This embodiment of the invention is prepared as follows:

Water Phase
Into a glycol-jacketed tank, add Purified Water, USP. Turn on scraper at 5 RPM. Add Pluronic F127. Chill to 5-10° C. (41-50° F.). Increase scraper speed to 10 RPM and continue mixing for twenty-four (24) hours. Add SD 40B Alcohol 190 Proof. Add Potato Starch. Add Germaben IIE. Continue mixing for one (1) hour.

Oil Phase
Into a 100-200 gallon heat-jacketed tank, add Isopropyl Palmitate. Add Lecithin. Without mixing, allow to dissolve for twenty-four (24) hours. Turn on mixer at 25 RPM. Add Arlacel 165. Add Cremophor GS32. Heat to 75-80° C. (167-176° F.).

Emulsion Phase

Turn on WATER PHASE mixer at 25 RPM. Add OIL PHASE to WATER PHASE. Add Simulgel NS. Increase scraper speed to 10 RPM and mix for 15 minutes.

### Example 3

A preferred embodiment of the invention for easy and ready to use hydrophilic drug actives was prepared containing the following ingredients:

| Ingredients | Amount | |
|---|---|---|
| Purified Water | 16.2542% | w/w |
| Lecithin 50% in Isopropyl Palmitate | 17.1800% | w/w |
| Polaxamer 407 30% in Purified Water | 45.8333% | w/w |
| Simulgel NS | 2.0000% | w/w |
| Glyceryl Stearate & PEG 100 Stearate | 0.7200% | w/w |
| Polyglyceryl 1-3 Distearate | 0.5000% | w/w |
| Germaben IIE | 1.0000% | w/w |
| Structure Solanace | 0.4000% | w/w |
| SD 40B Alcohol 190 Proof | 16.1125% | w/w |

This embodiment of the invention is prepared as follows:

Water Phase
Into a glycol-jacketed tank, add Purified Water, USP. Turn on scraper at 5 RPM. Add Pluronic F127. Chill to 5-10° C. (41-50° F.). Increase scraper speed to 10 RPM and continue mixing for twenty-four (24) hours. Add SD 40B Alcohol 190 Proof. Add Potato Starch. Add Germaben IIE. Continue mixing for one (1) hour.

Oil Phase
Into a 100-200 gallon heat-jacketed tank, add Isopropyl Palmitate. Add Lecithin. Without mixing, allow to dissolve for twenty-four (24) hours. Turn on mixer at 25 RPM. Add Arlacel 165. Add Cremophor GS32. Heat to 75-80° C. (167-176° F.).

Emulsion Phase
Turn on WATER PHASE mixer at 25 RPM. Add OIL PHASE to WATER PHASE. Add Simulgel NS. Increase scraper speed to 10 RPM and mix for 15 minutes.

### REFERENCES CITED

The following references, to the extent that they provide exemplary procedural or other details supplementary to those set forth herein, are specifically incorporated herein by reference.

### U.S. Patent Documents

U.S. Pat. No. 5,176,916 to Yamanaka, et al., issued Jan. 5, 1993
U.S. Pat. No. 5,613,958 to Kochinke, et al., issued Mar. 25, 1997
U.S. Pat. No. 5,665,378 to Davis, et al., issued Sep. 9, 1997
U.S. Pat. No. 5,837,289 to Grasela, et al., issued Nov. 17, 1998
U.S. Pat. No. 5,885,597 to Botknecht, et al., issued Mar. 23, 1999
U.S. Pat. No. 6,290,986 to Murdock, et al., issued Sep. 18, 2001

### REFERENCES

Alexandridis, P. Curr. Opin. Colloid Interface Sci. 1997, 2, 478
Nace, V. M., Nonionic Surfactants, Marcel-Dekker, New York 1996
Hvidt, S., et al. J. Phys. Chem. 1994, 98, 12320
Shawesh, A., et al. Pharmazie. 2002, 57(10):690-4
Timaksiz, F., et al. Pharmazie. 2005 60(7):518-23

What is claimed:

1. A lecithin organogel composition comprising:
a non-ionic emulsifying agent having a hydrophile-lipophile balance number of between about 8 to about 18;
a thickening agent;
a polyoxyethylene polyoxypropylene block copolymer; and
a lecithin.

2. The lecithin organogel composition of claim 1, wherein the non-ionic emulsifying agent comprises an oil-in-water emulsifier.

3. The lecithin organogel composition of claim 1, wherein the non-ionic emulsifying agent comprises a mono alkyl phosphate, a sorbitan ester, a polyethylene glycol oil/wax, a poly(ortho-ester)-fatty alcohol, a polyethylene glycol-stearate, a polysorbate, or a mixture thereof.

4. The lecithin organogel composition of claim 1, wherein said non-ionic emulsifying agent is in an amount ranging from about 0.1 to about 2.5 weight percent.

5. The lecithin organogel composition of claim 1, wherein the thickening polymer comprises an aqueous-alcoholic soluble thickener and has solubility in from about 1 percent to about 30 percent of alcohol.

6. The lecithin organogel composition of claim 1, wherein the thickening polymer comprises xanthan gum, acrylate/C₁₀-C₃₀ alkyl acrylate crosspolymer, hydroxypropylcellulose, acrylamide/sodium acryloyldimethyltaurate copolymer, isohexadecane and polysorbate 80, or a mixture thereof.

7. The lecithin organogel composition of claim 1, wherein said thickening agent is in an amount ranging from about 0.2 to about 5 weight percent.

8. A lecithin organogel composition comprising:
a non-ionic emulsifying agent having a hydrophile-lipophile balance number of between about 8 to about 18;
a thickening agent;
from about 1 weight percent to about 25 weight percent of a polyoxyethylene polyoxypropylene block copolymer; and
from about 1 weight percent to about 20 weight percent of a lecithin.

9. A lecithin organogel composition comprising:
a non-ionic emulsifying agent having a hydrophile-lipophile balance number of between about 8 to about 18;
a thickening agent;
from about 1 weight percent to about 25 weight percent of a polyoxyethylene polyoxypropylene block copolymer; and
from about 1 weight percent to about 20 weight percent of a lecithin.
wherein the content of the non-ionic emulsifying agent and the thickening polymer are included at a non-ionic emulsifying agent:thickening polymer ratio of from about 1:2 to 1:50.

10. A method for producing a transdermal delivery system for therapeutic agents comprising:
dissolving active therapeutic agents in the lecithin organogel composition of claim 1.

11. A method of dissolving an active drug into the lecithin organogel composition of claim 1 comprising:
using an electronic pestle and mortar to mix the active drug into the lecithin organogel composition.
